(19) United States
(12) Patent Application Publication
(10) Pub. No.: US 2004/0106785 A1
(43) Pub. Date: Jun. 3, 2004

(54) Inhibitors of RNA dependent RNA polymerase and uses thereof

(75) Inventors: Robert Deziel, Montreal (CA); Eric Fournier, Laval (CA); Arlene Roland, Castries (FR)

Correspondence Address: SEED INTELLECTUAL PROPERTY LAW GROUP PLLC, 701 FIFTH AVE, SUITE 6300, SEATTLE, WA, 98104-7092, US

(73) Assignee: Micrologix Biotech Inc., Vancouver (CA)

(21) Appl. No.: 10/628,086

(22) Filed: Jul. 25, 2003

Related U.S. Application Data
(60) Provisional application No. 60/398,426, filed on Jul. 25, 2002.

(51) Int. Cl. C07H 19/06; C07K 9/00

(52) U.S. Cl. 536/28.4; 530/322

(57) ABSTRACT

The invention herein provides novel viral DNA polymerase compounds. The invention also provides pharmaceutical compositions comprising such compounds, methods of using such compounds to treat diseases associated with viral DNA polymerase activity, and processes and intermediates useful for preparing such compounds.

### CROSS-REFERENCE TO RELATED APPLICATION

This application claims the benefit of U.S. Provisional Patent Application No. 60/398,426, filed Jul. 25, 2002, wherein this provisional application is incorporated herein by reference in its entirety.

### BACKGROUND OF THE INVENTION

1. Field of the Invention

The present invention relates generally to the treatment of viral infections, and more specifically, to methods and compounds for preparation and therapeutic use of anti-viral nucleoside derivatives, in addition to compositions of such anti-viral agents.

2. Description of the Related Art

Certain viral diseases, such as those caused by or associated with hepatitis C virus (HCV), are leading causes of liver disease, which can progress to cirrhosis and hepatocellular carcinoma (HCC) (Hoofnagle, Hepatology 26:15S, 1997). It is estimated that approximately 30,000 new cases of HCV infection occur every year in the U.S. alone (Kolykhalov et al., J. Virol. 74:2046, 2000), and of these, up to 85% may progress to chronic infection, which can result in serious diseases (including cirrhosis and HCC). Up to 10,000 people die each year from HCV related disease in the US, and over 170 million HCV carriers are estimated to exist worldwide. Existing treatments include interferon and ribavirin, but have only a 50% response rate in treated patients (Lindsay, Hepatology 26:71S, 1997; Reichard et al., Hepatology 26:108S, 1997).

HCV has a number of potential targets for therapeutics, including the non-structural protein 3 (NS3) protease, NS3 helicase, NS5B RNA dependent RNA polymerase (RdRP), and NS2/3 protease. For example, nucleotide analogs, whereby the internucleotide phosphate linkage has been replaced by an amide or ester, have been described (De Mesmaeker et al., U.S. Pat. No. 5,602,240) as 5-lipoxygenase inhibitors and a 4′-ethylamide of cytidine has also been described (Jung et al., Chem. Ber. 113:1775, 1980). However, RdRP appears to be a logical target because it does not exist in mammalian cells and it is essential for viral replication (Kolykhalov et al., J. Virol. 74:2046, 2000).

There are a number of different classes of molecules reported to be potential inhibitors of HCV RdRP. For example, WO 00/06529 discloses α,γ-diketoacids. Heterocycles and rhodanine derivatives are disclosed in WO 00/13708, WO 00/10573, and WO 00/18231. WO 01/47883, WO 02/04425, WO 03/010141, and WO 03/007945 all disclose substituted benzimidazoles. WO 03/026589 reports 4′-modified nucleosides. WO 01/90121 and WO 02/57425 report 2′-substituted nucleic acids. WO 02/06246 discloses dihydroxypyrimidine carboxylic acids. WO 02/057287 discloses 7-deaza-purine nucleosides. WO 01/77091 discloses heterocyclic compounds that covalently modify an important cysteine residue of RdRP. Some pyrimidine-based nucleoside 4-carboxylic acids are also known in the art (Jones et al., Carbohydr. Res. 1:187, 1965; Imai and Honjo, Chem. Pharm. Bull. 13:7, 1965; Mansour et al., EP 515156, 1992; Misaki, FR 2488618, 1981; Jung, et al., Chem. Ber. 113(5):1775, 1980; Schinazi et al., J. Med. Chem. 21:1141, 1978; Marx et al., EP 799834, 1996) . Nucleosides disclosed thus far with activity against RdR polymerases (such as HCV NS5B RdRP) have been modified at various positions, such as the 4′ and 2′ positions (but all have a 5—OH group).

Nucleoside analogs have also been identified for other indications. For example, nucleoside 4′-acids, 4′-amides, and 4′-peptidyinucleotides based on purine, uracil and thymidine nucleosides have been disclosed as vasodilation agents and anti-inflammatory agents (Stein et al., U.S. Pat. No. 4,029,884, 1977), as nucleoside biosynthesis inhibitors (Baker, Tetrahedron 30:2939, 1974; Henn et al., J. Med. Chem. 36:1570, 1993; Elliot et al., J. Med. Chem. 31:250, 1988); and as adenosine triphosphate receptor interacting agents (Kawana et al., J. Org. Chem. 37:288, 1972; Inaki et al., Nucleosides and Nucleotides 17:1, 1988; Pehk et al., Bioorg. Med. Chem. Lett. 7:2159, 1997).

Thus, a need exists for identifying and developing anti-viral agents having improved activity and reduced toxicity (and do not cause other undesirable side effects), particularly therapeutics for the treatment of HCV. The present invention meets such needs, and further provides other related advantages.

### BRIEF SUMMARY OF THE INVENTION

The present invention generally provides nucleoside derivatives, in particular, RNA-dependent RNA polymerase (RdRP) inhibitors, and compositions of such compounds for use in treating or preventing, for example, viral infections such as those caused by hepatitis C virus (HCV). In particular, the present invention is directed to nucleoside analogues and derivatives thereof having unexpectedly high inhibitory activity against HCV replication, particularly against NS5B RdRP.

In one aspect, the present invention comprises a compound or a pharmaceutically acceptable salt thereof according to structure (I):

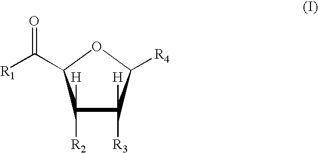

wherein:

R₁ is OH, N alkyl, one or more amino acids; each of R₂ and R₃ is independently OH, N₃, hydrogen, halogen, alkyl, alkoxy, amine, or absent and linked via an alkene; and R₄ is a substituted or unsubstituted heterocycle.

In another aspect of the invention, the above disclosed compounds and pharmaceutically acceptable salts thereof and pharmaceutical compositions thereof are used to treat or prevent a disease wherein the disease is (a) caused or associated with a virus, such as HBV, Influenza, HCV or HIV of any type; or (b) caused by or associated with DNA or RNA processing enzymes, such as NS5B RdRP from any type of HCV. In certain embodiments of the invention, the above disclosed compounds and pharmaceutically acceptable salts thereof, or pharmaceutical compositions are used in combination with one or more inhibitors selected from a nucleoside reverse transcriptase inhibitor, a non-nucleoside reverse transcriptase inhibitor, a helicase inhibitor, an RNaseH inhibitor, a kinase inhibitor, a protease inhibitor and a polymerase inhibitor.

In another aspect of the invention, the above disclosed compounds and pharmaceutically acceptable salts thereof, or pharmaceutical compositions are used to modulate, inhibit, or abrogate the activity of an RNA or DNA processing enzyme, such as HCV NS5B RdRP.

### DETAILED DESCRIPTION OF THE INVENTION

As set forth above, the present invention provides compositions and methods for using and making anti-viral nucleoside analogues, and derivatives thereof, to treat or prevent viral diseases. In particular, these nucleoside analogues, and derivatives thereof, are useful for treating or preventing viral infections, such as hepatitis C virus (HCV) infections. The invention, therefore, relates generally to the surprising discovery that certain nucleoside analogues, and derivatives thereof, have an unexpectedly high activity against HCV. Accordingly, the compounds of the invention are useful, for example, as a tool for in vitro and cell-based assays for the biological mechanisms of HCV infection (e.g., replication and transmission), and are useful as potential therapeutics for the prevention or treatment of HCV infection and potentially HCV related disease. More specifically, the invention provides viral RNA dependent RNA polymerase (RdRP) inhibitor compounds and pharmaceutical compositions comprising such compounds, methods of using such compounds to treat diseases associated with viral RdRP activity (e.g., inhibitors of NS5B RdRP of HCV), and processes and intermediates useful for preparing such compounds. Discussed in more detail below are such nucleoside analogues and derivatives thereof, suitable for use within the present invention, as well as representative compositions and therapeutic uses thereof.

Prior to setting forth the invention in more detail, it may be helpful to an understanding thereof to set forth definitions of certain terms to be used hereinafter.

In the present description, any concentration range, percentage range, or integer range is to be understood to include the value of any integer within the recited range and, when appropriate, fractions thereof (such as one tenth and one hundredth of an integer), unless otherwise indicated. As used herein, “about” or “comprising essentially of” mean ±15%. The use of the alternative (e.g., “or”) should be understood to mean either one, both or any combination thereof of the alternatives. In addition, it should be understood that the individual compounds, or groups of compounds, derived from the various combinations of the structures and substituents described herein, are disclosed by the present application to the same extent as if each compound or group of compounds was set forth individually. Thus, selection of particular structures or particular substituents is within the scope of the present invention.

The amino acid designations are herein set forth as either the standard one- or three-letter code. Polar amino acids include asparagine (Asp or N) and glutamine (Gln or Q); as well as basic amino acids such as arginine (Arg or R), lysine (Lys or K), histidine (His or H), and derivatives thereof; and acidic amino acids such as aspartic acid (Asp or D) and glutamic acid (Glu or E), and derivatives thereof. Hydrophobic amino acids include tryptophan (Trp or W), phenylalanine (Phe or F), isoleucine (Ile or I), leucine (Leu or L), methionine (Met or M), valine (Val or V), and derivatives thereof; as well as other non-polar amino acids such as glycine (Gly or G), alanine (Ala or A), proline (Pro or P), and derivatives thereof. Amino acids of intermediate polarity include serine (Ser or S), threonine (Thr or T), tyrosine (Tyr or Y), cysteine (Cys or C), and derivatives thereof. Unless specified otherwise, amino acids may be in either the D- or L-configuration. A capital letter indicates an L-enantiomer amino acid; a small letter indicates a D-enantiomer amino acid. Some exemplary modified amino acids may include phenylglycine (Phg), 2,3-diamino butyric acid (Dab), 2,3-diamino propionic acid (Dap), β-methylaspartate (MeAsp), cyclohexylalanine (β-Cha), citrulline (Cit) norleucine (Nle), norvaline (Nvl), isonipecotic acid (Ina), pipecolic acid (homoproline) (Pip or hpro), p-aminophenylacetic acid (Apa), 2-aminobutyric acid (Abu), sarcosine (Sar or N-methyl glycine or MeGly), 6-amino-hexanoic acid (Ahx), 3- or 4-mercaptoproline derivatives, N5-acetyl-N5-hydroxy-L-ornithine, and α-N-hydroxyamino acids. A nucleoside analog or derivative thereof may include any one or combination of the above-noted natural and non-natural amino acids (including, e.g., peptides having up to 20 amino acids), or any other modification known in the art.

As used herein, the term “alkyl” refers to a saturated or unsaturated, branched, straight-chain or cyclic monovalent hydrocarbon group derived by the removal of one hydrogen atom from a single carbon atom of a parent alkane, alkene or alkyne. Typical alkyl groups include methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl , prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl , but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like.

The term “alkyl” is specifically intended to include straight- or branched-hydrocarbons having from 1 to 25 carbon atoms, more preferably 5 to 20, and most preferably 10 to 18. The alkyls may have any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. When a specific level of saturation is intended, the expressions “alkanyl,” “alkenyl,” and “alkynyl” are used. The expression “lower alkyl” refers to alkyl groups comprising from 1 to 8 carbon atoms. The alkyl group may be substituted or unsubstituted.

“Alkanyl” refers to a saturated branched, straight-chain or cyclic alkyl group. Typical alkanyl groups include methanyl; ethanyl; propanyls such as propan-1-yl, propan-2-yl (isopropyl), cyclopropan-1-yl, etc.; butyanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

“Alkenyl” refers to an unsaturated branched, straight-chain, cyclic alkyl group, or combinations thereof having at least one carbon-carbon double bond derived by the removal of one hydrogen atom from a single carbon atom of a parent alkene. The group may be in either the cis or trans conformation about the double bond(s). Typical alkenyl groups include ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl ; butenyls such as but-1-en-1-yl, but-1-en-2-yl, 2-methyl-prop-1-en-1-yl, but-2-en-1-yl , but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like. The alkenyl group may be substituted or unsubstituted.

“Alkynyl” refers to an unsaturated branched, straight chain or cyclic alkyl group having at least one carbon-carbon triple bond derived by the removal of one hydrogen atom from a single carbon atom of a parent alkyne. Typical alkynyl groups include ethynyl; propynyls such as prop-1-yn-1-yl, prop-2-yn-1-yl, etc.; butynyls such as but-1-yn-1-yl, but-1-yn-3-yl, but-3-yn-1-yl , etc.; and the like.

“Alkyidiyl” refers to a saturated or unsaturated, branched, straight-chain or cyclic divalent hydrocarbon group derived by the removal of one hydrogen atom from each of two different carbon atoms of a parent alkane, alkene or alkyne, or by the removal of two hydrogen atoms from a single carbon atom of a parent alkane, alkene or alkyne. The two monovalent radical centers or each valency of the divalent radical center can form bonds with the same or different atoms. Typical alkyldiyl groups include methandiyl; ethyldiyls such as ethan-1,1-diyl, ethan-1,2-diyl, ethen-1,1-diyl, ethen-1,2-diyl; propyldiyls such as propan-1,-diyl, propan-1,2-diyl, propan-2,2-diyl, propan-1,3-diyl, cyclopropan-1,1-diyl, cyclopropan-1,2-diyl, prop-1-en-1,1-diyl, prop-1-en-1,2-diyl, prop-2-en-1,2-diyl, prop-1-en-1,3-diyl, cycloprop-1-en-1,2-diyl, cycloprop-2-en-1,2-diyl, cycloprop-2-en-1,1-diyl, prop-1-yn-1,3-diyl, etc.; butyldiyls such as, butan-1,1-diyl, butan-1,2-diyl, butan-1,3-diyl, butan-1,4-diyl, butan-2,2-diyl, 2-methyl-propan-1,1-diyl, 2-methyl-propan-1,2-diyl, cyclobutan-1,1-diyl; cyclobutan-1,2-diyl, cyclobutan-1,3-diyl, but-1-en-1,1-diyl, but-1-en-1,2-diyl, but-1-en-1,3-diyl, but-1-en-1,4-diyl, 2-methyl-prop-1-en-1,1-diyl, 2-methanylidene-propan-1,1-diyl, buta-1,3-dien-1,1-diyl, buta-1,3-dien-1,2-diyl, buta-1,3-dien-1,3-diyl, buta-1,3-dien-1,4-diyl, cyclobut-1-en-1,2-diyl, cyclobut-1-en-1,3-diyl, cyclobut-2-en-1,2-diyl, cyclobuta-1,3-dien-1,2-diyl, cyclobuta-1,3-dien-1,3-diyl, but-1-yn-1,3-diyl, but-1-yn-1,4-diyl, buta-1,3-diyn-1,4-diyl, etc.; and the like. When a specific level of saturation is intended, the nomenclature alkanyldiyl, alkenyldiyl or alkynyldiyl is used. In preferred embodiments, the alkyldiyl group is (C₁-C₄) alkyldiyl. Also preferred are saturated acyclic alkanyidiyl groups in which the radical centers are at the terminal carbons, e.g., methandiyl (methano); ethan-1,2-diyl (ethano); propan-1,3-diyl (propano); butan-1,4-diyl (butano); and the like (also referred to as alkylenos, defined infra).

“Alkyleno” refers to a straight-chain alkyldiyl group having two terminal monovalent radical centers derived by the removal of one hydrogen atom from each of the two terminal carbon atoms of straight-chain parent alkane, alkene or alkyne. Typical alkyleno groups include methano; ethylenos such as ethano, etheno, ethyno; propylenos such as propano, prop[1]eno, propa[1,2]dieno, prop[1]yno, etc.; butylenos such as butano, but[1]eno, but[2]eno, buta[1,3]dieno, but[1]yno, but[2]yno, but[1,3]diyno, etc.; and the like. When a specific level of saturation is intended, the nomenclature alkano, alkeno or alkyno is used. In preferred embodiments, the alkyleno group is (C₁-C₆) or (C₁-C₄) alkyleno. Also preferred are straight-chain saturated alkano groups, e.g., methano, ethano, propano, butano, and the like.

“Heteroalkyl, Heteroalkanyl, Heteroalkenyl, Heteroalkanyl, Heteroalkyldiyl and Heteroalkyleno” refer to alkyl, alkanyl, alkenyl, alkynyl, alkyldiyl and alkyleno groups, respectively, in which one or more of the carbon atoms (and any associated hydrogen atoms) are each independently replaced with the same or different heteroatoms or heteroatomic groups. Typical heteroatoms or heteroatomic groups that can be included in these groups include —O—, —S—, —Se—, —O—O—, —S—S—, —O—S—, —O—S—O—, —O—NR′—, —NR′—, —NR′—NR′—, ═N—N═, —N═N—, —N═N—NR′—, —PH—, —P(O)₂—, —O—P(O)₂—, —SH₂—, —S(O)₂—, —SnH₂— and the like, and combinations thereof, including —NR′—S(O)₂—, where each R′ is independently selected from hydrogen, alkyl, alkanyl, alkenyl, alkynyl, aryl, arylalkyl, heteroaryl and heteroarylalkyl, as defined herein.

“Aryl” refers to a monovalent aromatic hydrocarbon group derived by the removal of one hydrogen atom from a single carbon atom of a parent aromatic ring system. Typical aryl groups include groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In preferred embodiments, the aryl group is (C₅-C₁₄) aryl, with (C₅-C₁₀) being even more preferred. Particularly preferred aryls are cyclopentadienyl, phenyl and naphthyl. The aryl group may be substituted or unsubstituted.

“Arylalkyl” refers to an acyclic alkyl group in which one of the hydrogen atoms bonded to a carbon atom, typically a terminal or sp³ carbon atom, is replaced with an aryl group. Typical arylalkyl groups include benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylakenyl or arylalkynyl is used. In preferred embodiments, the arylalkyl group is (C₆-C₂₀) arylalkyl, e.g., the alkanyl, alkenyl or alkynyl moiety of the arylalkyl group is (C₁-C₆) and the aryl moiety is (C₅-C₁₄). In particularly preferred embodiments the arylalkyl group is (C₆-C₁₃), e.g., the alkanyl, alkenyl or alkynyl moiety of the arylalkyl group is (C₁-C₃) and the aryl moiety is (C₅-C₁₀).

“Heteroaryl” refers to a monovalent heteroaromatic group derived by the removal of one hydrogen atom from a single atom of a parent heteroaromatic ring system, which may be monocyclic or fused ring (i.e., rings that share an adjacent pair of atoms). Typical heteroaryl groups include groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In preferred embodiments, the heteroaryl group is a 5-14 membered heteroaryl, with 5-10 membered heteroaryl being particularly preferred. The most preferred heteroaryl groups are those derived from thiophene, pyrrole, benzothiophene, benzofuran, indole, pyridine, quinoline, imidazole, oxazole and pyrazine. The heteroaryl group may be substituted or unsubstituted.

“Heteroalicyclic” refers to a monocyclic or fused ring group having in the ring(s) one or more atoms selected preferably from nitrogen, oxygen and sulfur. The rings may also have one or more double bonds. However, the rings do not have a completely conjugated π-electron system. The heteroalicyclic ring may be substituted or unsubstituted. When substituted, the substituted group(s) preferably are selected independently from alkyl, aryl, haloalkyl, halo, hydroxy, alkoxy, mercapto, cyano, sulfonamidyl, aminosulfonyl, acyl, acyloxy, vitro, and substituted amino.

“Heteroarylalkyl” refers to an acyclic alkyl group in which one of the hydrogen atoms bonded to a carbon atom, typically a terminal or sp³ carbon atom, is replaced with a heteroaryl group. When one or more specific alkyl moiety is intended, the nomenclature heteroarylalkanyl, heteroarylakenyl or heterorylalkynyl is used. In preferred embodiments, the heteroarylalkyl group is a 6-20 membered heteroarylalkyl, e.g., the alkanyl, alkenyl or alkynyl moiety of the heteroarylalkyl is 1-6 membered and the heteroaryl moiety is a 5-14-membered heteroaryl. In particularly preferred embodiments, the heteroarylalkyl is a 6-13 membered heteroarylalkyl, e.g., the alkanyl, alkenyl or alkynyl moiety is 1-3 membered and the heteroaryl moiety is a 5-10 membered heteroaryl.

“Cycloalkyl” encompasses cyclic alkyl groups that contain between 3 and 8 carbon atoms and have a single cyclic ring, illustrated by cyclopropyl, cyclobutyl, cyclopentyl, and cyclooctyl. The cycloalkyl ring may be substituted or unsubstituted. Again, a substituted cycloalkyl ring carries one or more substituent groups, independently selected preferably from alkyl, aryl, haloalkyl, halo, hydroxy, alkoxy, mercapto, cyano, sulfonamidyl, aminosulfonyl, acyl, acyloxy, vitro, and substituted amino.

“Halogen” or “halo” refers to fluoro (F), chloro (Cl), bromo (Br), iodo (I). As used herein, -X refers to independently any halogen.

“Acyl” group refers to the C(O)—R″ group, where R″ is selected preferably from hydrogen, hydroxy, alkyl, haloalkyl, cycloalkyl, aryl optionally substituted with one or more alkyl, haloalkyl, alkoxy, halo and substituted amino groups, heteroaryl (bonded through a ring carbon) optionally substituted with one or more alkyl, haloalkyl, alkoxy, halo and substituted amino groups and heteroalicyclic (bonded through a ring carbon) optionally substituted with one or more alkyl, haloalkyl, alkoxy, halo and substituted amino groups. Acyl groups include aldehydes, ketones, acids, acid halides, esters and amides. Preferred acyl groups are carboxy groups, e.g., acids and esters. Esters include amino acid ester derivatives. The acyl group may be attached to a compound's backbone at either end of the acyl group, i.e., via the C or the R″. When the acyl group is attached via the R″, then C will bear another substituent, such as hydrogen, alkyl, and the like.

“Substituted” refers to a group in which one or more hydrogen atoms are each independently replaced with the same or different substituent(s). Typical substituents include -X, —R¹³, —O—, ═O, —OR, —SR¹³, —S—, ═S, —NR¹³R¹³, ═NR¹³, CX₃, —CF₃, —CN, —OCN, —SCN, —NO, NO₂, ═N₂, —N₃, —S(O)₂O—, —S(O)₂OH, —S(O)₂R¹³, —OS(O₂)O—, —OS(O)₂OH, —OS(O)₂R¹³, —P(O)(O⁻)₂, —P(O)(OH)(O⁻), —OP(O)₂(O⁻), —C(O)R¹³, —C(S)R¹³, —C(O)OR¹³, —C(O)O—, —C(S)OR¹³, and —C(NR¹³)NR¹³R¹³, wherein each X is independently a halogen; each R¹³ is independently hydrogen, halogen, alkyl, aryl, arylalkyl, arylaryl, arylheteroalkyl, heteroaryl, heteroarylalkyl NR¹⁴R¹⁴, —C(O)R¹⁴, and —S(O)₂R¹⁴; and each R¹⁴ is independently hydrogen, alkyl, alkanyl, alkynyl, aryl, arylalkyl, arylheteralkyl, arylaryl, heteroaryl or heteroarylalkyl.

“Prodrug” herein refers to a compound that is converted into the parent compound in vivo. Prodrugs often are useful because, in some situations, they may be easier to administer than the parent compound. For example, the prodrug may be bioavailable by oral administration while the parent compound is not. The prodrug may also have improved solubility in pharmaceutical compositions over the parent compound. An example of a prodrug would be a compound of the embodiments of the present invention that is administered, for example, as an ester (the “prodrug”) to facilitate transmittal across a cell membrane when water solubility is detrimental to mobility, but then is metabolically hydrolyzed to an active entity once inside the cell where water solubility is beneficial. Such a compound is generally inactive (or less active) until converted to the active form.

“Pharmaceutically acceptable salt” refers to a salt of a compound of the invention that is pharmaceutically acceptable and that possesses the desired pharmacological (e.g., anti-viral) activity. Such salts include the following: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine, and the like.

RNA-Dependent RNA Polymerase Inhibitors

As set forth above, the present invention provides nucleoside analogues and derivatives thereof, pharmaceutically acceptable salts thereof, and therapeutic uses thereof. In particular, the nucleoside analogues of the instant invention have unexpectedly high anti-viral activity, particularly against HCV. More specifically, the present invention relates to compounds that inhibit RdRP. Preferably, the nucleoside compounds of the present invention inhibit RdRP of any type of HCV (also known as non-structural protein 5B, NS5B). Preferred RdRP inhibitors are those of structure (I):

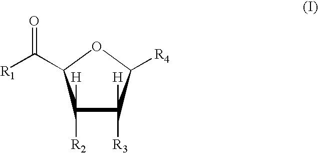

wherein R₁ is OH, N-alkyl, one or more amino acids; each of R₂ and R₃ is independently OH, N₃, hydrogen, halogen, alkyl, alkoxy, amine or absent via a linked alkene; and R₄ is a heterocycle of any composition; or pharmaceutically acceptable salts, compositions, and prodrugs thereof.

Other preferred compounds of the present invention are those of structure (II):

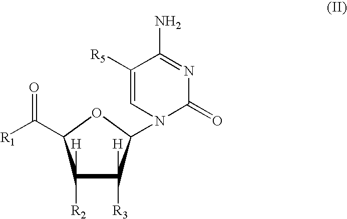

wherein R₁ is OH, N-alkyl, one or more amino acids; each of R₂ and R₃ are independently OH, N₃, hydrogen, halogen, alkyl, alkoxy, or amine; and R₅ is OH, N₃, hydrogen, halogen, alkyl, alkoxy, or amine; or pharmaceutically acceptable salts, compositions, or prodrugs thereof.

Still other preferred compounds of the present invention are those of structure (III):

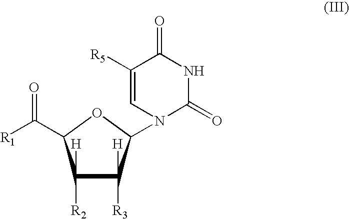

wherein R₁ is OH, N-alkyl, one or more amino acids; each of R₂ and R₃ is independently OH, N₃, hydrogen, halogen, alkyl, alkoxy, amines; and R₅ is OH, N₃, H, halogen, alkyl, alkoxy, or amine; or pharmaceutically acceptable salts, compositions, or prodrugs thereof.

Certain preferred compounds of structure (II) are those wherein R₁ is OH; each of R₂, R₃ and R₅ is independently OH, N₃, hydrogen, halogen, alkyl, alkoxy or amines; or pharmaceutically acceptable salts, compositions, or prodrugs thereof. Examples of particularly preferred R₁ substituents for compounds of structure (II) are one or more natural or non-natural amino acids known in the art, or two or more natural or non-natural amino acids of any composition (e.g., peptides of up to 20 amino acids); or pharmaceutically acceptable salts, compositions, or prodrugs thereof. Examples of particularly preferred R₂, R₃ and R₅ substituents for compounds of structure (II) are each independently OH, N₃, hydrogen, halogen, alkyl, alkoxy or amines; or pharmaceutically acceptable salts, compositions, or prodrugs thereof.

In another preferred embodiment, the present invention comprises compounds of structure (IV):

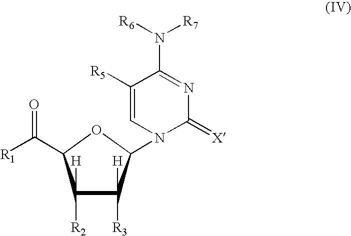

wherein R₁ is OH, N-alkyl, one or more amino acids; R₂ and R₃ are each independently OH, N₃, hydrogen, halogen, alkyl, alkoxy, amines; and R₅ is OH, N₃, hydrogen, halogen, alkyl, alkoxy, amines; R₆ and R₇ are each independently hydrogen, alkyl, aryl, aralkyl, heteroalkyl, heteroaryl, acyl, or closed into a ring encompassing the nitrogen they are attached to optionally substituted with alkyl, heteroalkyl, heteroaryl, or aryl groups; and X′ is O, NH, S, C(—R₈)—R₉, or N—R₈, wherein each of R₈ and R₉ is independently hydrogen, alkyl, aryl, aralkyl, heteroalkyl, heteroaryl, or acyl; or a pharmaceutically acceptable salt thereof.

Therapeutic Formulations and Methods of Use

As described herein, compounds of the instant invention show surprising and exceptionally strong inhibition of viral replication, particularly HCV. Compounds of the invention, including the compounds of structures (I) to (IV), exhibit anti-viral activity against HCV. Certain compounds exhibit anti-viral activity against HCV, including compounds according to structural formula (II) wherein R₁ is OH, each of R₂ and R₃ is OH, and R₅ is hydrogen, with an IC₅₀ below 10 μM in an HCV RdR polymerase assay (see Example 17). In certain embodiments, the invention provides compounds capable of inhibiting viral replication, preferably HCV, at clinically relevant concentrations.

The invention also relates to pharmaceutical compositions that contain one or more compounds used to treat or prevent a viral infection (e.g., HCV). The invention further relates to methods for treating or preventing viral infections by administering to a subject a nucleoside analogue or derivative thereof of the instant invention, or a mixture of such compounds at a dose sufficient to treat or prevent a viral infection, as described herein. The nucleoside analogues and derivatives thereof, or cocktail of such compounds, are preferably part of a pharmaceutical composition when used in the methods of the present invention.

In preferred embodiments of the invention, nucleoside compounds described herein are used to treat or prevent a viral infection in a subject, preferably the subject is a mammal, even more preferably the subject is a human. In other preferred embodiments, the viral infection is (a) caused or associated with one or more viruses, such as any type of HBV, influenza, HCV, or HIV; or (b) caused or associated with a DNA or RNA processing enzyme, such as NS5B RdRP from HCV. Certain compounds of the instant invention, including the compound of the structure (II), will preferably show good overall biopharmaceutical properties and will be orally available. In one preferred embodiment, the invention comprises a pharmaceutical composition comprising a nucleoside anti-viral compound as described herein (or a pharmaceutically active derivative thereof and a pharmaceutically acceptable carrier, excipient or diluent. Preferably, the pharmaceutical composition comprises an anti-viral compound that has structure (II). The term “pharmaceutically active derivative” refers to any compound that, upon administration to a subject in need thereof, is capable of providing directly or indirectly (e.g., a pro-drug) the compounds of the instant invention.

As set forth herein, the active compound may be included in a pharmaceutically acceptable carrier or diluent for administration to a subject in need thereof in an amount effective to treat or prevent an HCV infection. A preferred dose of the active compound for all of the above-mentioned indications is in a range from about 0.01 mg/kg to about 300 mg/kg per day; preferably about 0.1 mg/kg to about 100 mg/kg per day, more preferably about 0.5 mg/kg to about 25 mg/kg body weight of the recipient per day. A typical topical dosage will range from about 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to a person having ordinary skill in the art. The compound can be conveniently administered in any suitable unit dosage form, including one containing from about 1 mg to about 3000 mg, and preferably about 5 mg to about 500 mg of active ingredient per unit dosage. In one preferred embodiment, an oral dosage of about 1 mg to about 500 mg, preferably about 10 mg to about 250 mg, and more preferably about 25 mg to about 250 mg is administered to a subject to treat or prevent a viral infection.

The active ingredient should be administered to achieve peak plasma concentrations of the active compound of about 0.001 μM to about 30 μM, and preferably about 0.01 μM to about 10 μM. This may be achieved, for example, by intravenous injection of a composition or formulation of a nucleoside analogue or derivative thereof of the invention, optionally in saline or other aqueous medium. In another embodiment, a nucleoside analogue or derivative thereof of the invention or composition thereof is administered as a bolus.

The concentration of active compound in a pharmaceutical composition of the instant invention will depend on absorption, distribution, inactivation, and excretion rates of the drug, as well as other factors known to those of skill in the art. It is to be understood that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

Oral compositions will generally include an inert diluent or an edible carrier. They may be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate or Sterores; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier, such as a fatty oil. In addition, dosage unit forms can contain various other materials which modify the physical form of the dosage unit, for example, coatings of sugar, shellac, or enteric agents. See generally “Remington's Pharmaceutical Sciences,” Mack Publishing Co., Easton, Pa.

Pharmaceutically acceptable carriers suitable for use with a composition of the present invention may include, for example, a thickening agent, a buffering agent, a solvent, a humectant, a preservative, a chelating agent, an adjuvant, and the like, and combinations thereof. Pharmaceutically acceptable carriers for therapeutic use are well known in the pharmaceutical art, and as described herein and, for example, in Remington's Pharmaceutical Sciences, Mack Publishing Co. (A. R. Gennaro, ed., 18^{th} Edition, 1990) and in CRC Handbook of Food, Drug, and Cosmetic Excipients, CRC Press LLC (S. C. Smolinski, ed., 1992).

The pharmaceutical composition of the instant invention will preferably include at least one of a pharmaceutically acceptable vehicle, carrier, diluent, or excipient, in addition to one or more nucleoside analogue or derivative thereof of the invention and, optionally, other components. A composition of the invention may have a variety of active ingredients, such as a nucleoside compound, or a cocktail of two or more nucleoside compounds, or a cocktail of one or more nucleoside compounds or derivatives thereof with one or more antibiotic, antifungal, anti-inflammatory, or other anti-viral compound. Preferably, nucleoside compounds of the instant invention are used in combination with one or more inhibitors selected from a nucleoside reverse transcriptase inhibitor, a non-nucleoside reverse transcriptase inhibitor, a helicase inhibitor, an RNaseH inhibitor, a kinase inhibitor, a protease inhibitor and a polymerase inhibitor.

Solutions or suspensions used for parenteral, intradermal, subcutaneous, or topical application can include the following components: a sterile diluent such as water for injection, saline solution, fixed oils, polyethylene glycols, glycerin, propylene glycol or other synthetic solvents; anti-bacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic. If administered intravenously, preferred carriers are physiological saline or phosphate buffered saline (PBS) or an adjuvant. Exemplary adjuvants are alum (aluminum hydroxide, REHYDRAGEL®), aluminum phosphate, virosomes, liposomes with and without Lipid A, Detox (Ribi/Corixa), MF59, or other oil and water emulsions type adjuvants, such as nanoemulsions (see, e.g., U.S. Pat. No. 5,716,637) and submicron emulsions (see, e.g., U.S. Pat. No. 5,961,970), and Freund's complete and incomplete. In one preferred embodiment, a pharmaceutical composition of the invention is sterile.

In certain embodiments, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. For example, as is known in the art, some of these materials can be obtained commercially from Alza Corporation (CA) and Gilford Pharmaceuticals (Baltimore, Md.).

Liposomal suspensions may also be pharmaceutically acceptable carriers. These may be prepared according to methods known to those skilled in the art, for example, as described in U.S. Pat. No. 4,522,811. For example, liposome formulations may be prepared by dissolving appropriate lipid(s) (such as stearoyl phosphatidyl ethanolamine, stearoyl phosphatidyicholine, arachadoyl phosphatidylcholine, and cholesterol) in an inorganic solvent that is then evaporated, leaving behind a thin film of dried lipid on the surface of the container. An aqueous solution of the active compound or its monophosphate, diphosphate, and/or triphosphate derivatives are then introduced into the container. The container is then swirled by hand to free lipid material from the sides of the container and to disperse lipid aggregates, thereby forming the liposomal suspension.

In one preferred embodiment, nucleoside compounds of the invention and pharmaceutically acceptable salts thereof, or pharmaceutical compositions are used to modulate, inhibit, or abrogate the activity of an RNA or DNA processing enzyme, such as HCV NS5B RdRP. Preferably, any the above disclosed compounds and pharmaceutically acceptable salts thereof or pharmaceutical compositions are used to modulate, inhibit, or abrogate the activity of an RNA or DNA processing enzyme in order to treat or treat a viral infections, such those caused by or associated with HCV.

The invention having been described, the following examples are intended to illustrate, and not limit, the invention.

### EXAMPLES

In the examples below, the following abbreviations have the following meanings. Any abbreviations not defined have their generally accepted meaning. Unless otherwise stated, all temperatures are in degrees Celsius.

| | |
|---|---|
| DMSO = | dimethyl sulfoxide |
| EtOAc = | ethyl acetate |
| TFA = | trifluoroacetic acid |
| THF = | tetrahydrofuran |
| MeOH = | methanol |
| DMTr = | dimethoxy trityl |
| DCC = | 1,3-dicyclohexylcarbodiimide |
| DMAP = | 4-(dimethylamino)pyridine |
| DMF = | dimethyl formamide |
| DIEA = | N,N-diisopropylethylamine |
| NaOMe = | sodium methoxide |
| TEMPO = | 2,2,6,6-tetramethyl-1-piperidinyloxy |
| BAIB = | iodobenzene diacetate |

General: Unless noted otherwise, reagents, starting material and solvents were purchased from commercial suppliers (Aldrich, Fluka, Sigma, and etc.), and used without further purification; reactions were run under nitrogen atmosphere; reaction mixtures were monitored by thin layer chromatography (silica TLC), analytical high performance liquid chromatography (anal. HPLC), or mass spectrometry; reaction mixtures were commonly purified by flash column chromatography on silica gel, or by preparative HPLC using the general protocol described below; NMR samples were dissolved in deuterated solvent (CD3OD, CDCl₃, or DMSO-d6), and spectra were acquired with a Varian Gemini 2000 instrument (500 MHz) under standard parameters; and mass spectrometric identification was performed by an electrospray ionization method (ES-MS) with a Waters 2690 Alliance System.

### Example 1

### BENZOIC ACID 5-(4-BENZOYLAMINO-2-OXO-2H-PYRIMIDIN-1-YL)-2-[BIS-(4-METHOXY-PHENYL)-PHENYL-METHOXYMETHYL]-TETRAHYDRO-FURAN-3-YL ESTER

To 10 g (16 mmol) of 5′-DMTr-Bz-dC in 40 mL CH₂Cl₂ was added 1.8 mL (24 mmol) pyridine, 10 mg of DMAP, and 1.6 mL (19.2 mmol) benzoyl chloride. After 3 hours at room temperature (RT), the mixture was partly concentrated in vacuo, filtered through a bed of SiO₂ and concentrated in vacuo to give the title compound (intermediate A) without further purification.

### Example 2

### BENZOIC ACID 5-(4-BENZOYLAMINO-2-OXO-2H-PYRIMIDIN-1-YL)-2-HYDROXYMETHYL-TETRAHYDRO-FURAN-3-YL ESTER

To 12.7 g (17 mmol) of intermediate A in 40 mL CH₂Cl₂ was added 5 mL TFA. After 2 hours at RT, toluene was added and the mixture was concentrated in vacuo. The concentrate was purified by silica gel chromatography (600 mL silica gel, eluted with EtOAc/MeOH: 20:1-20:3) to give the title compound (intermediate B) as a white solid (5.4 g, 80%). m/z calcd for C₂₃H₂₁N₃O₆ (M+H)⁺, 435.43, found 436.18.

### Example 3

### 5-(4-BENZOYLAMINO-2-OXO-2H-PYRIMIDIN-1-YL)-3-BENZOYLOXY-TETRAHYDRO-FURAN-2-CARBOXYLIC ACID

To 237 mg (0.54 mmol) of intermediate B in 5 mL CH₂Cl₂ was added 19 mg (0.12 mmol) TEMPO and 369 mg (1.14 mmol) BAIB. After 4 hours at RT, 1 drop of 50% aqueous CH₃CN was added; 2 hours later the mixture was concentrated to a fine white powder, suspended in EtOAc, collected by filtration, and washed with EtOAc to give the title compound (intermediate C) as a white solid (227 mg, 90%). ¹H NMR (DMSO; 500 MHz): d 2.4 (m, 1H, H₂′), d 2.75 (m, 1H, H₂′), d 4.85 (m, 1H, H₄′), d 5.8 (d, 1H, H₃′), d 6.4 (m, 1H, H₁′), d 7.4-7.65 (m, 6H, Ar), d 7.7 (d, 1H, CH═CH), d 8.05, (m, 4H, Ar), d 8.65, (d, 1H, CH═CH), d 11.25 (s, 1H, NH). m/z calcd for C₂₃H₁₉N₃O₇ (M+H)⁺, 449.41, found 450.13.

### Example 4

### 5-(4-AMINO-2-OXO-2H-PYRIMIDIN-1-YL)-3-HYDROXY-TETRAHYDRO-FURAN-2-CARBOXYLIC ACID

To 25 mg (0.1 mmol) of intermediate C in 1 mL MeOH was added to 1 mL 0.5M NaOMe in MeOH. After 2 hours of vigorous stirring at RT, Amberlite IR50SH+ was added in portion to neutral pH. The resin was filtered, washed with MeOH, and the filtrate concentrated to dryness. The concentrate was dissolved in water, purified on 30 g of C₁₈. The product was analyzed by analytical LC-MS, and freeze-dried to give the title compound 1 as a white powder (11 mg, 90%). Retention time (anal. HPLC: XTerra MS C18, 5 μm, 4.6×150 mm, A: DMHA 20 mM, pH 7.58; B: MeOH; 0 min 100% A; 20 min 80%B, 1 mL/min, UV at 250-260 nm): 11.56 min. m/z calcd for C₉H₁₁N₃O₅ (M+H)⁺, 241.2, found 242.08.

### Example 5

### 2-{[5-(4-AMINO-2-OXO-2H-PYRIMIDIN-1-YL)-3-HYDROXY-TETRAHYDRO-FURAN-2-CARBONYL]-AMINO}-3-METHYL-PENTANOIC ACID METHYL ESTER

To 35 mg (0.08 mmol) of intermediate C in 1 mL DMF was added 1 mg DMAP, 16.48 mg (0.08 mmol) DCC, 15 μL (0.08 mmol) DIEA, and 18 mg (0.1 mmol) L-isoleucine methyl ester hydrochloride. After 12 hours of vigorous stirring at RT, the mixture was concentrated to dryness, dissolved in 1 mL EtOAc, filtered through 5 mL of silica gel and further eluted with 2 mL EtOAc. The solution was concentrated and 1 mL of 0.25 M NaOMe was added. After 2 hours of vigourous stirring at RT, Amberlite IR50SH+ was added in portion to neutral pH. The resin was filtered, washed with MeOH, and the filtrate concentrated to dryness. The concentrate was purified by silica gel chromatography (5 mL silica gel, dissolved in 1 mL 10% MeOH in EtOAc, washed with 1 mL EtOAc and eluted with 3 mL dichloromethane/MeOH/water: 7:3:1) to give the title compound 2 as a white powder (12.2 mg, 60%). Retention time (anal. HPLC: XTerra MS C18, 5 μm, 4.6×150 mm, A: 5 mM ammonium formate in water; B: 5 mM ammonium formate in MeOH; 0 min 100% A; 20 min 80%B, 0.3 mL/min, UV at 250-260 nm): 16.07 min. m/z calcd for C₁₆H₄₁N₄O₆ (M−H)⁻, 368.38, found 367.19.

### Example 6

### 2-{[5-(4-AMINO-2-OXO-2H-PYRIMIDIN-1-YL)-3-HYDROXY-TETRAHYDRO-FURAN-2-CARBONYL]-AMINO}-3-METHYL-PENTANOIC ACID

Compound 2 (10 mg, 0.03 mmol) was treated in the manner described in Example 4 to give the title compound 3 as a white powder (8 mg, 90%). Retention time (anal. HPLC: XTerra MS C18, 3.5 μm, 3.0×50 mm, A: 5 mM ammonium formate in water; B: 5 mM ammonium formate in MeOH; 0 min 100% A; 20 min 80%B, 0.3 mL/min, UV at 250-260 nm): 12.99 min. m/z calcd for C₁₅H₂₂N₄O₆ (M−H)⁻, 354.36, found 353.

### Example 7

### 2-{[5-(4-AMINO-2-OXO-2H-PYRIMIDIN-1-YL)-3-HYDROXY-TETRAHYDRO-FURAN-2-CARBONYL]-AMINO}4-METHYL-PENTANOIC ACID METHYL ESTER

Intermediate C and L-Leucine methyl ester hydrochloride were treated in a manner similar to that described in Example 5 to give the title compound 4. m/z calcd for C₁₆H₂₄N₄O₆ (M−H)⁻, 368.38, found 367.2. Retention time (anal. HPLC): 16.21 min.

### Example 8

### 6-AMINO-2-{[5-(4-AMINO-2-OXO-2H-PYRIMIDIN-1-YL)-3-HYDROXY-TETRAHYDRO-FURAN-2-CARBONYL]-AMINO}-HEXANOIC ACID METHYL ESTER

Intermediate C and L-Lysine-Bz methyl ester hydrochloride were treated in a manner similar to that described in Example 5 to give the title compound 5. m/z calcd for C₁₆H₂₅N₅O₆ (M+H)⁺, 383.40, found 384.18. Retention time (anal. HPLC): 11.46 min.

### Example 9

### 2-{[5-(4-AMINO-2-OXO-2H-PYRIMIDIN-1-YL)-3-HYDROXY-TETRAHYDRO-FURAN-2-CARBONYL]-AMINO}-4-METHYLSULFANYL-BUTYRIC ACID METHYL ESTER

Intermediate C and L-Methionine methyl ester hydrochloride were treated in a manner similar to that described in Example 5 to give the title compound 6. m/z calcd for C₁₅H₂₂N₄O₆S(M−H)⁻, 386.42, found 385.16. Retention time (anal. HPLC): 14.69 min.

### Example 10

### 2-{[5-(4-AMINO-2-OXO-2H-PYRIMIDIN-1-YL)-3-HYDROXY-TETRAHYDRO-FURAN-2-CARBONYL]-AMINO}-3-PHENYL-PROPIONIC ACID METHYL ESTER

Intermediate C and L-Phenylalanine methyl ester hydrochloride were treated in a manner similar to that described in Example 5 to give the title compound 7. m/z calcd for Cl₉H₂₂N₄O₆(M−H)⁻, 402.40, found 401.21. Retention time (anal. HPLC): 16.27 min.

### Example 11

### 2-{[5-(4-AMINO-2-OXO-2H-PYRIMIDIN-1-YL)-3-HYDROXY-TETRAHYDRO-FURAN-2-CARBONYL]-AMINO}-PHENYL-ACETIC ACID METHYL ESTER

Intermediate C and L-Phenylglycine methyl ester hydrochloride were treated in a manner similar to that described in Example 5 to give the title compound 8. m/z calcd for C₁₈H₂₀N₄O₆(M−H)⁻, 388.37, found 387.18. Retention time (anal. HPLC): 15.78 min.

### Example 12

### 2-{[5-(4-AMINO-2-OXO-2H-PYRIMIDIN-1-YL)-3-HYDROXY-TETRAHYDRO-FURAN-2-CARBONYL]-AMINO}-4-METHYL-PENTANOIC ACID

Compound 7 was treated in a manner similar to that described in Example 4 to give the title compound 9. m/z calcd for C₁₅H₂₂N₄O₆(M−H)⁻, 354.36, found 353.2. Retention time (anal. HPLC): 13.14 min.

### Example 13

### 6-AMINO-2-{[5-(4-AMINO-2-OXO-2H-PYRIMIDIN-1-YL)-3-HYDROXY-TETRAHYDRO-FURAN-2-CARBONYL]-AMINO}-HEXANOIC ACID

Compound 8 was treated in a manner similar to that described in Example 4 to give the title compound 10. m/z calcd for C₁₆H₂₅N₅O₆(M+H)⁺, 369.37, found 370.15. Retention time (anal. HPLC): 2.88 min.

### Example 14

### 2-{[5-(4-AMINO-2-OXO-2H-PYRIMIDIN-1-YL)-3-HYDROXY-TETRAHYDRO-FURAN-2-CARBONYL]-AMINO}-4-METHYLSULFANYL-BUTYRIC ACID

Compound 9 was treated in a manner similar to that described in Example 4 to give the title compound 11. m/z calcd for C₁₄H₂₀N₄O₆S (M−H)⁻, 372.40, found 371.14. Retention time (anal. HPLC): 12.07 min.

### Example 15

### 2-{[5-(4-AMINO-2-OXO-2H-PYRIMIDIN-1-YL)-3-HYDROXY-TETRAHYDRO-FURAN-2-CARBONYL]-AMINO}-3-PHENYL-PROPIONIC ACID

Compound 10 was treated in a manner similar to that described in Example 4 to give the title compound 12. m/z calcd for C₁₈H₂₀N₄O₆ (M−H)⁻, 388.37, found 387.18. Retention time (anal. HPLC): 13.89 min.

### Example 16

### 2-{[5-(4-AMINO-2-OXO-2H-PYRIMIDIN-1-YL)-3-HYDROXY-TETRAHYDRO-FURAN-2-CARBONYL]-AMINO}-PHENYL-ACETIC ACID

Compound 11 was treated in a manner similar to that described in Example 4 to give the title compound 13.

### Example 17

### HEPATITIS C POLYMERASE ASSAY

Compounds of the instant invention were tested for inhibitory activity against the HCV RdR polymerase according to the following assay description. The HCV RdR polymerase assay measures the ability of this enzyme to catalyze the incorporation of ribonucleotides into a nascent strand of RNA (RNA template) and can be used to determine the activity of the potential antiviral compounds. To investigate the activity of the compounds described in this invention, two HCV RdR polymerase assays were used, one using non-radioactive ribonucleotides (assay obtained from Replizyme Ltd., York, UK) and another using ³²P-UTP. The latter is performed as follows: Test compounds are dissolved in DMSO or appropriate solvent, diluted to the desired concentration in water and then 5 μL each compound is transferred to a well of a microtiter plate on ice. A recombinant HCV polymerase (1 mg, 25 μL) is then added, followed by 20 μL of reaction mix that typically contains a template-primer poly-(rA)-p(dT)₁₂₋₁₈ with a 1-7 ratio of template and primers and poly A to 70 nM, 5 units of RNase inhibitor (Rnasin Promega), 1 μCi of α32P-UTP (0.1-0.2 μL) (3000 Ci/mmol), 20 μM UTP, 5 μL of 10× reaction buffer (200 mM Tris-HCl pH7.0, 10 mM DTT, MgCl₂ 20 mM or MnCl₂ 2 mM, 50 mM NaCl in DEPC-treated water. After incubation at 30° C. for a desired time (i.e., 60 minutes) the neosynthesized RNAs were aggregated to sonicated salmon sperm DNA by addition of 50 μL of 0.5 mg salmon sperm DNA (Gibco-Life technologies) per mL, and then precipitated with TCA (using 100 μL 20% TCA, 1% PPᵢ and then incubated at 4° C. for 30 minutes). 180 μL of each precipitate is transferred to a Millipore MultiScreen plate well and thereby filtered onto DEAE paper filters using a MultiScreen Separation System (Millipore). The precipitate is washed 3 times with 1% TCA/0.1% PPᵢ. The filters are dried at 42° C. for 15 minutes and then transferred using the Multiscreen Multipunch system (Millipore) into scintillation vials for liquid scintillation counting after addition of 5 mL of scintillation liquid to each vial. Alternatively, an appropriate plate reader can be used.

TABLE 1

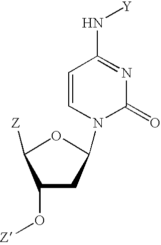

Intermediate Compounds

| Intermediate | | | |
|---|---|---|---|
| Number | Z′ | Y | Z |
| A | Bz if Z is H | —CH₂—O-DMTr | H if X is Bz |
| | (or H if Z is Bz) | | (or Bz if X is H) |
| B | Bz | —CH₂—OH | Bz |
| C | Bz | —COOH | Bz |

TABLE 2

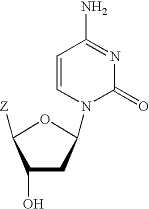

Methyl Esters and Final Compounds

| Compound | | Compound | |
|---|---|---|---|
| Number | Z | Number | Z |
| 2 | —C(═O)-L-Ile-OMe | 3 | —C(═O)-L-Ile |
| 4 | —C(═O)-L-Leu-OMe | 9 | —C(═O)-L-Leu |
| 5 | —C(═O)-L-Lys-OMe | 10 | —C(═O)-L-Lys |
| 6 | —C(═O)-L-Met-OMe | 11 | —C(═O)-L-Met |
| 7 | —C(═O)-L-Phe-OMe | 12 | —C(═O)-L-Phe |
| 8 | —C(═O)-L-Phg-OMe | 13 | —C(═O)-L-Phg |
| | | 1 | —COOH |

All of the U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing description, although specific embodiments of the invention have been described herein for purposes of illustration, one of ordinary skill in the art can easily ascertain the essential characteristics of this invention, and without departing from the spirit and scope thereof, can make various changes and modifications of the invention without undue experimentation. Accordingly, the invention is not limited except as by the appended claims.

1. A compound and pharmaceutically acceptable salts thereof according to structural formula (I): 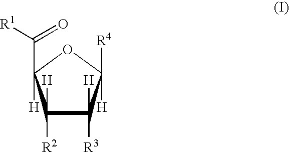
wherein:
R¹ is OH, N-alkyl, one or more amino acids;
each of R² and R³ is independently OH, N₃, hydrogen, halo, alkyl, alkoxy, amines or absent and linked via an alkene; and
R⁴ is a heterocycle.

2. The compound of claim 1 and pharmaceutically acceptable salts thereof according to structural formula (II): 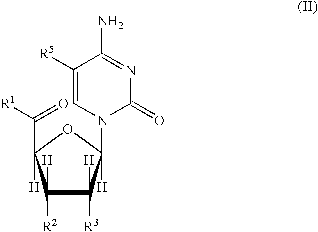
wherein:
R¹ is OH, N-alkyl, one or more amino acids;
each of R² and R³ is independently OH, N₃, hydrogen, halo, alkyl, alkoxy, or amine; and
R⁵ is OH, N₃, hydrogen, halo, alkyl, alkoxy, or amine.

3. The compound of claim 1 and pharmaceutically acceptable salts thereof according to structural formula (III): 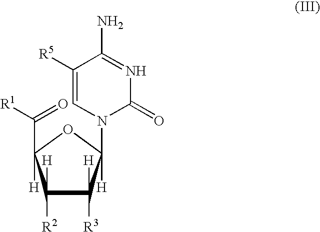
wherein:
R¹ is OH, N-alkyl, one or more amino acid;
each of R² and R³ is independently OH, N₃, hydrogen, halo, alkyl, alkoxy, or amine; and
R⁵ is OH, N₃, hydrogen, halo, alkyl, alkoxy, or amine.

4. A pharmaceutical composition comprising a compound according to any one of claims 1-3, and a pharmaceutically acceptable carrier, diluent, or excipient.

5. A method of treating or preventing a disease or condition associated with DNA or RNA processing enzymes, comprising administering to a subject in need thereof a pharmaceutical composition of claim 4 in an amount effective to treat or prevent said disease or condition associated with DNA or RNA processing enzymes.

6. The method of claim 5 wherein the disease or condition is a viral disease.

7. The method of claim 6 wherein the viral disease is caused by hepatitis C virus (HCV) or human immunodeficiency virus (HIV).

8. The method of claim 5 wherein said composition further comprises a second therapeutic agent.

9. The method of claim 5 wherein said composition further comprises a second therapeutic agent selected from the group consisting of an inhibitor of nucleoside reverse transcriptase, a non-nucleoside reverse transcriptase inhibitor, a helicase inhibitor, an RNase H inhibitor, a kinase inhibitor, and a protease inhibitor.

10. A method of modulating the activity of a DNA or RNA processing enzymes, the method comprising contacting a DNA or RNA processing enzyme with a modulatory amount of a compound or composition as described in any one of claims 1-4.

11. The method of claim 10 wherein the DNA or RNA processing enzyme being modulated is RNA-dependent RNA polymerase.

12. The method of claim 11 wherein the RNA-dependent RNA polymerase is non-structural protein 5B from HCV.
